(19) United States
(12) Patent Application Publication
(10) Pub. No.: US 2019/0274702 A1
(43) Pub. Date: Sep. 12, 2019

(54) CYSTOTHROMBUS AND GASTROENTERO-THROMBUS EVACUATORS

(71) Applicant: Vincent Sutliff, III, Baltimore, MD (US)

(72) Inventor: Vincent Sutliff, III, Baltimore, MD (US)

(21) Appl. No.: 15/914,608

(22) Filed: Mar. 7, 2018

(51) Int. Cl. A61B 17/32; A61B 1/00; A61B 1/273; A61B 1/307

(52) U.S. Cl. CPC A61B 17/32002; A61B 1/0014; A61B 2017/00296; A61B 1/307; A61B 1/2736

(57) ABSTRACT

Thrombus evacuator devices are provided. A cystothrombus provides an aspirator tube fluidly communicating to a suction intake, wherein a maceration blade is provided within the aspirator tube adjacent to the suction intake. A rotational element us operatively associated to the maceration blade for morcellating blood clots urged through the suction intake, wherein the aspirator tube is dimensioned to slidably nest in a cystoscope sheath adapted to be inserted into a human urethra. In another embodiment, a gastroentro-thrombus evacuator includes a double lumen tube having non-fluidly connected first and second lumens terminating into a coupling cavity of a coupling device removably attached to a distal end of the double lumen tube. The coupling device provides a suction intake and a flushing outlet fluidly communicating with the coupling cavity. A rotating maceration blade is operational adjacent to the suction intake, while a suction is urged through the first lumen.

### BACKGROUND OF THE INVENTION

The present invention relates to medical devices and, more particularly, to thrombus evacuators adapted to simultaneously aspirate and morcellate blood clots from the human urinary bladder (cystothrombus evacuator) and the human digestive system (gastroentero-thrombus evacuator).

The presence of blood clots in the urinary bladder, either from spontaneous bleeding or following surgery, may cause acute bladder outflow obstruction, which is a urological emergency.

The presence of blood clots in the digestive system, either from spontaneous bleeding or following surgery, obscures the source of bleeding, interfering with diagnostic and therapeutic gastrointestinal endoscopy, delaying definitive treatment.

As can be seen, there is a need for thrombus evacuators enabling simultaneous aspiration and morcellation of blood clots from the human urinary bladder (cystothrombus evacuator) and the human digestive system (gastroentero-thrombus evacuator).

### SUMMARY OF THE INVENTION

In one aspect of the present invention, a cystothrombus evacuator device includes an aspirator tube extending from a distal end to a proximal end; a suction intake fluidly communicating with the distal end; a maceration blade provided within the aspirator tube adjacent to the suction intake; and rotational element operatively associated to the maceration blade for morcellating blood clots urged through the suction intake, wherein the aspirator tube is dimensioned to slidably nest in a cystoscope sheath adapted to be inserted into a human urethra.

In another aspect of the present invention, a gastroentero-thrombus evacuator includes a double lumen tube extending from a distal end to a proximal end, the double lumen tube providing non-fluidly connected first and second lumens; a coupling device removably attached to the distal end, the coupling device providing an endo opening for operatively engaging a distal end of an endoscope; the coupling device providing a suction intake fluidly communicating with the first lumen; the coupling device providing a flushing outlet fluidly communicating with the second lumen; a maceration blade provided adjacent to the suction intake; and rotational element operatively associated to the maceration blade for morcellating blood clots urged through the suction intake, the rotational element extending through the second lumen.

In yet another aspect of the present invention, the gastroentero-thrombus evacuator includes a double lumen tube extending from a distal end to a proximal end, the double lumen tube providing non-fluidly connected first and second lumens; a coupling device removably attached to the distal end, the coupling device providing an endo opening for operatively engaging a distal end of an endoscope; the coupling device providing a coupling cavity fluidly interconnecting the first and second lumens; the coupling device providing a suction intake fluidly communicating with the coupling cavity; the coupling device providing a flushing outlet fluidly communicating with the coupling cavity; a maceration blade provided adjacent to the suction intake; and rotational element operatively associated to the maceration blade for morcellating blood clots urged through the suction intake, the rotational element extending through the second lumen.

These and other features, aspects and advantages of the present invention will become better understood with reference to the following drawings, description and claims.

### BRIEF DESCRIPTION OF THE DRAWINGS

FIG. 1 is an elevational view of an exemplary embodiment of a cystothrombus evacuator of the present invention, shown in transparency for clarity purposes;

FIG. 2 is a schematic elevational view of an exemplary embodiment of a cystothrombus evacuator of the present invention, shown in use;

FIG. 3 is an elevational view of an exemplary embodiment of a cystothrombus evacuator of the present invention, shown in transparency for clarity, adapted for the purposes of, including but not limited to, coupling to a cystoscope sheath;

FIG. 4 is an elevational view of an exemplary embodiment of a cystothrombus evacuator of the present invention, shown in transparency for clarity, adapted for purposes of, including but not limited to, coupling to a cystoscope sheath and flexible video cystoscope;

FIG. 5 is an elevational view of an exemplary embodiment of a gastroentero-thrombus evacuator of the present invention shown in transparency for clarity;

FIG. 6 is an elevational view of an exemplary embodiment of a gastroentero-thrombus evacuator of the present invention adapted for coupling to the tip of a video endoscope;

FIG. 7 is an elevational view of an exemplary embodiment of a gastroentero-thrombus evacuator of the present invention including a video input device;

FIG. 8 is a schematic elevational view of an exemplary embodiment of a gastroentero-thrombus evacuator of the present invention including a video input device, shown in use;

FIG. 9 is a detail elevational view of an exemplary embodiment of a distal end of the gastroentero-thrombus evacuator and the coupling device of the present invention;

FIG. 10 is a detail perspective view of an exemplary embodiment of the distal end of the gastroentero-thrombus evacuator attached to the tip of a video endoscope;

FIG. 11 is two cooperating detail perspective and front views of an exemplary embodiment of the suction tip of the present inventions;

FIG. 12 is two cooperating detail perspective and front views of an exemplary embodiment of an alternative suction tip a maceration/homogenization tip of the present inventions;

FIG. 13 is two cooperating detail perspective and front views of an exemplary embodiment of an alternative suction tip of the present inventions;

FIG. 14 is two cooperating detail perspective and front views of an exemplary embodiment of a maceration/homogenization blade of the present invention;

FIG. 15 is two cooperating detail perspective and front views of an exemplary embodiment of an alternative maceration/homogenization blade of the present inventions; and

FIG. 16 is two cooperating detail perspective and front views of an exemplary embodiment of a whisk configuration of an alternative maceration/homogenization blade of the present inventions.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a cystothrombus evacuator device having an aspirator tube fluidly communicating to a suction intake, wherein a maceration blade is provided within the aspirator tube adjacent to the suction intake. A rotational element is operatively associated to the maceration blade for morcellating blood clots urged through the suction intake, wherein the aspirator tube is dimensioned to slidably nest in a cystoscope sheath adapted to be inserted into a human urethra. In another embodiment, a gastroentero-thrombus evacuator includes a double lumen tube having non-fluidly connected first and second lumens terminating into a coupling cavity of a coupling device removably attached to the tip of a video endoscope. The coupling device provides a suction intake and a flushing outlet fluidly communicating with the coupling cavity. A rotating maceration blade is operational adjacent to the suction intake, while a suction is urged through the first lumen.

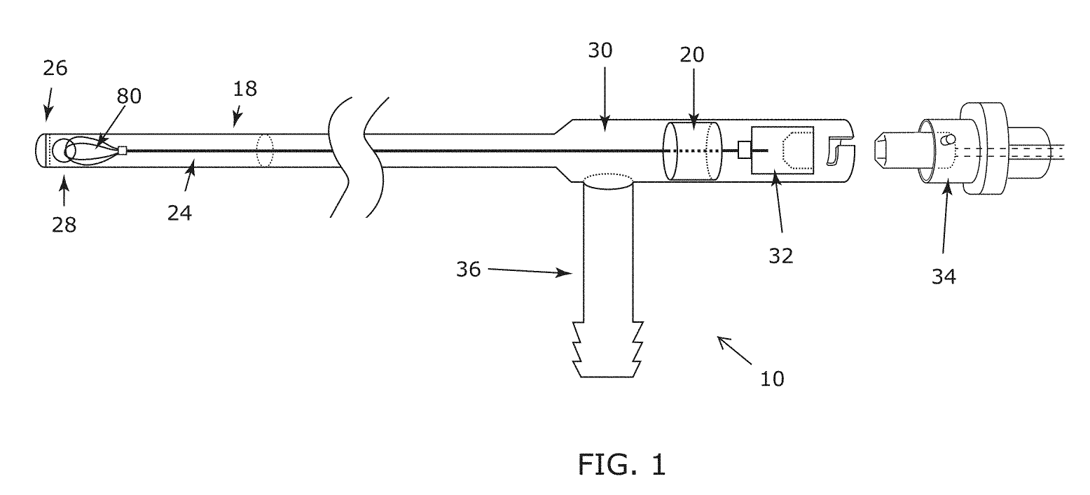

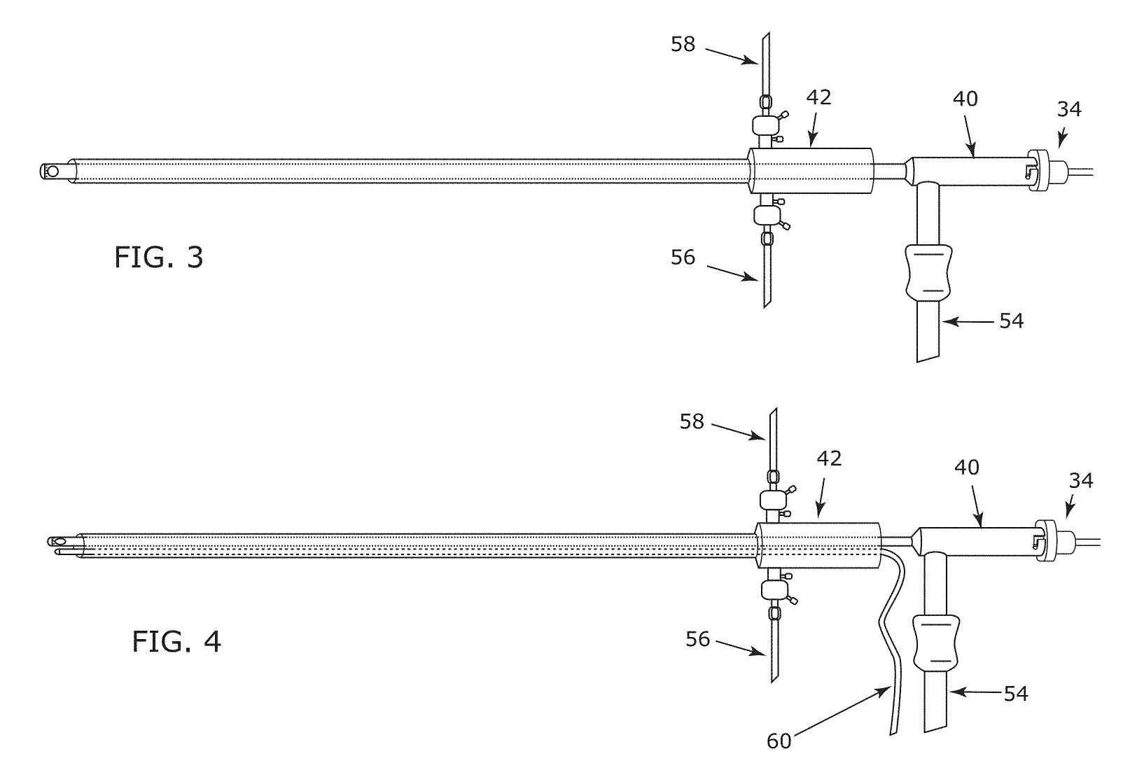

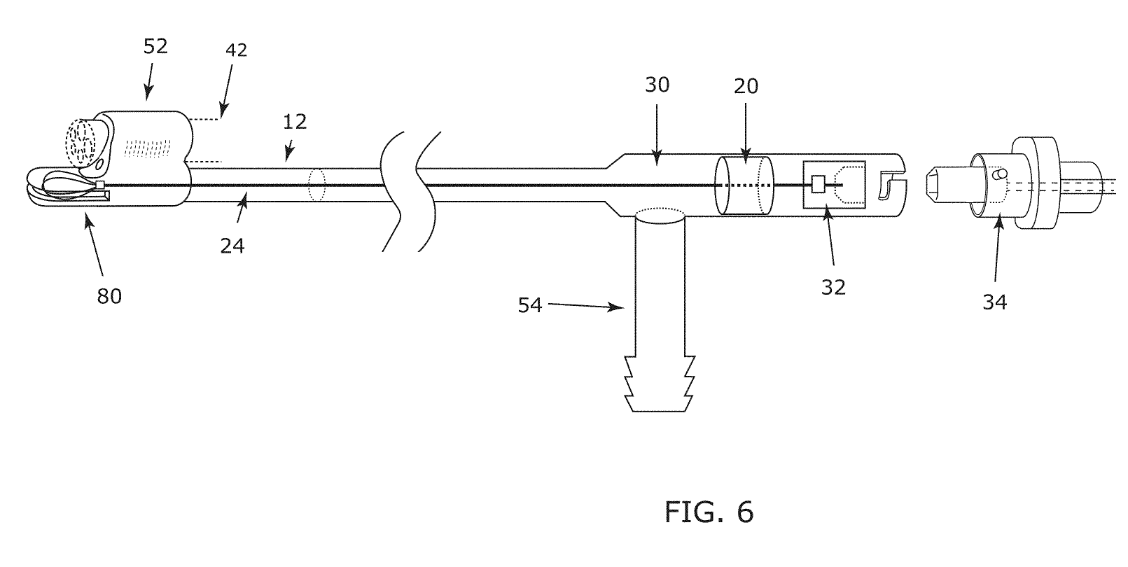

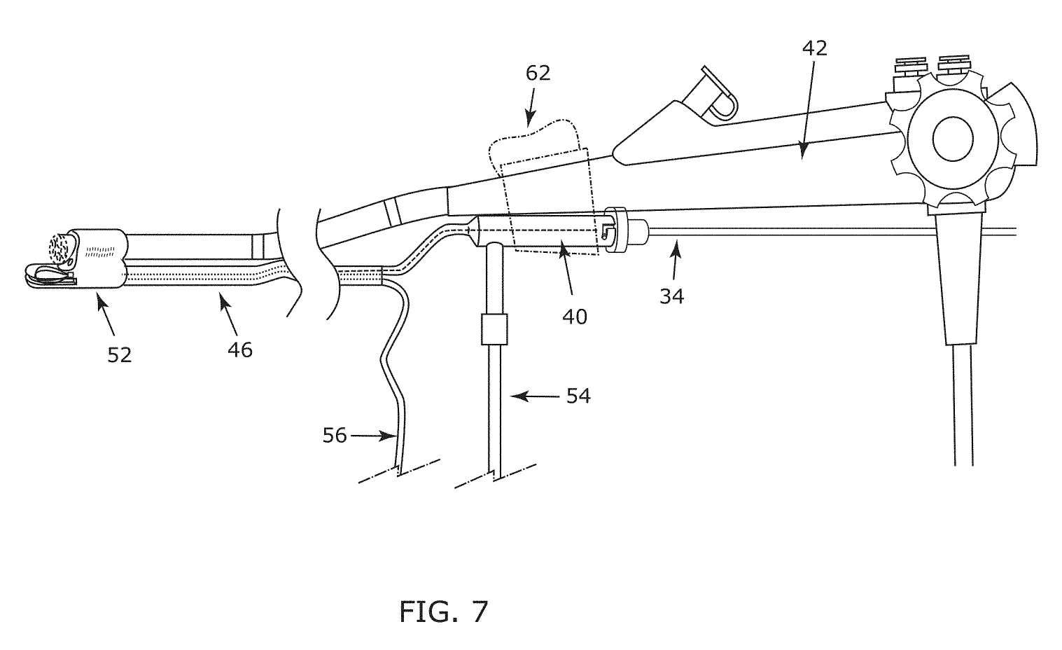

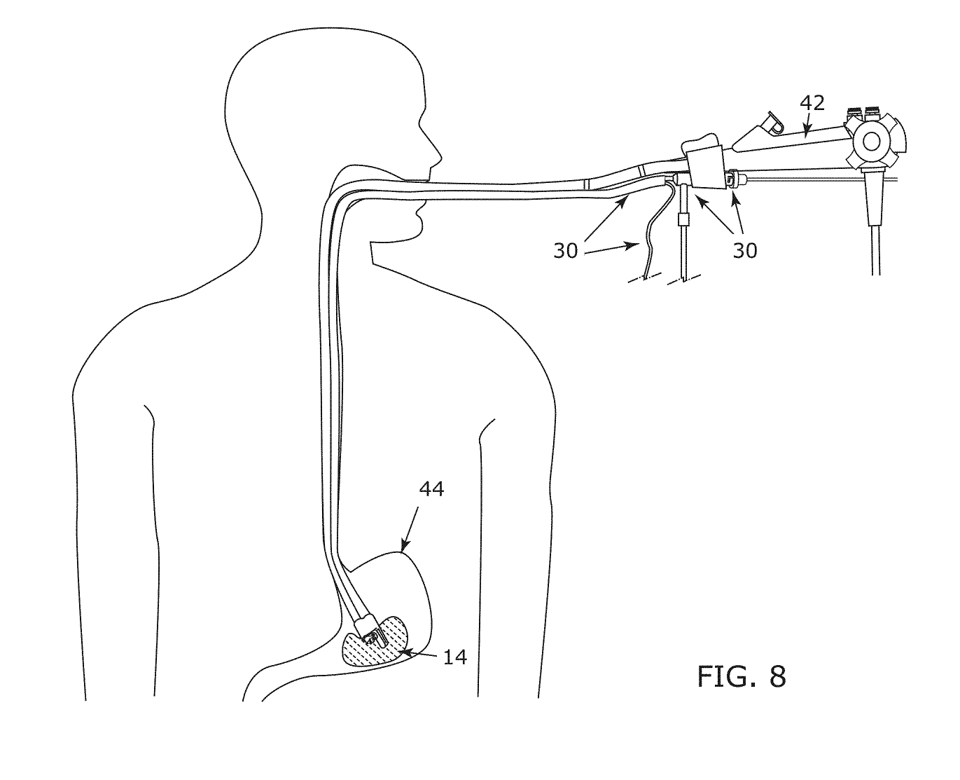

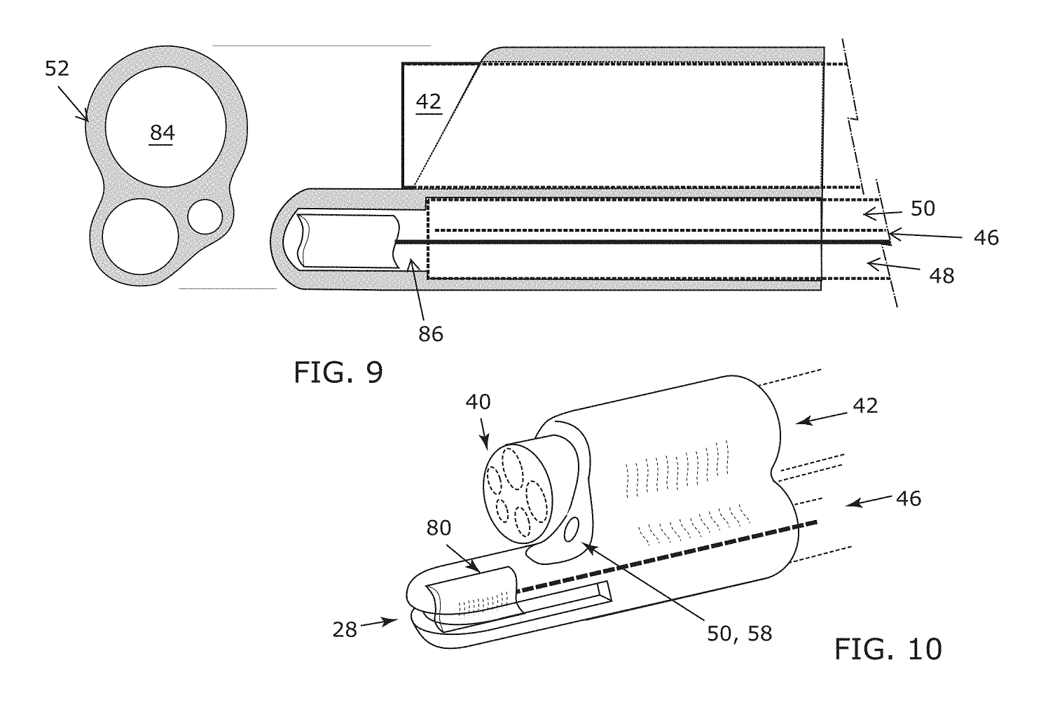

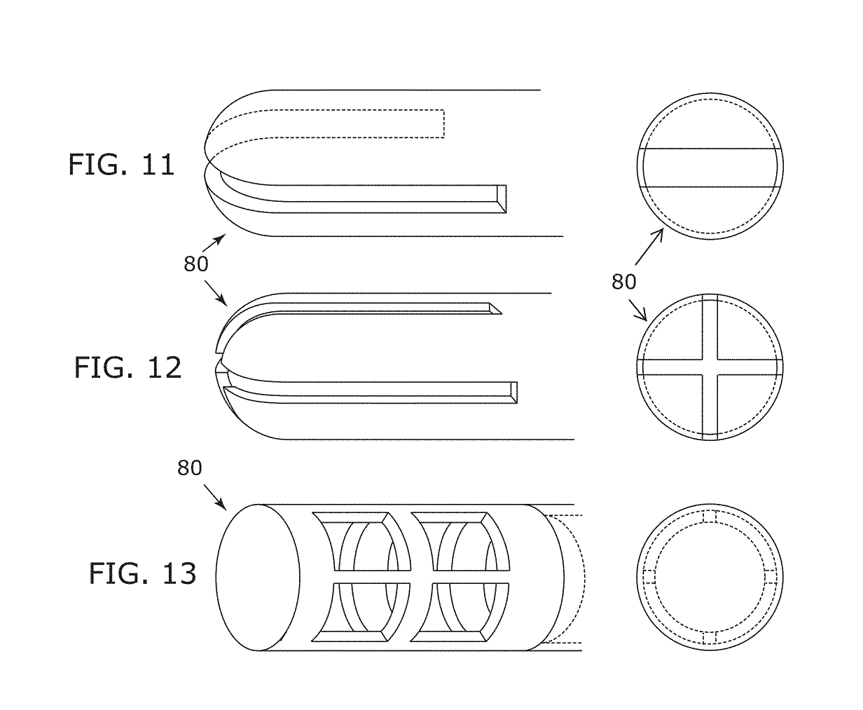

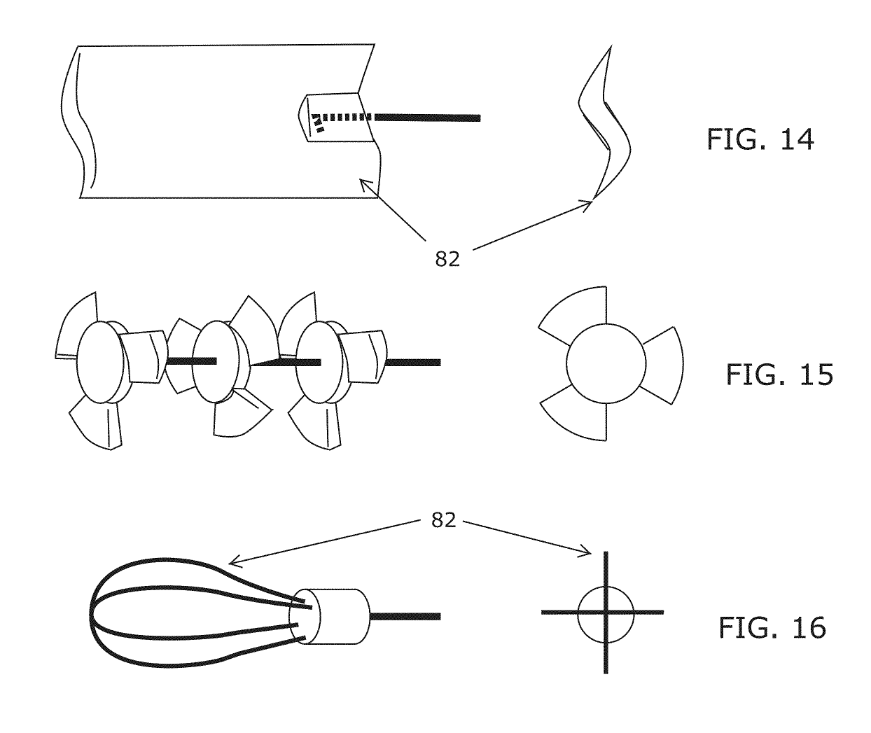

Referring to FIGS. 1 through 16, the present invention may include thrombus evacuators for removing blood clots 14 from the human urinary bladder (cystothrombus evacuator 10) and the human upper digestive system (gastroentero-thrombus evacuator 30).

Referring to FIGS. 1 through 4, the urinary bladder cystothrombus evacuator 10 may be a medical device operatively associated through a cystoscope instrument 12 for the emergent removal of organized blood clots 14 from the human urinary bladder 16, rapidly reversing bladder outflow obstruction. The cystoscope 12 (such as a catheter) is an instrument adapted to be inserted into the urethra for examining the urinary bladder 16. The cystoscope 12 provides a therapeutic cystoscope sheath 22 through which the cystothrombus evacuator 10 is afforded access to the patient's urinary bladder 16.

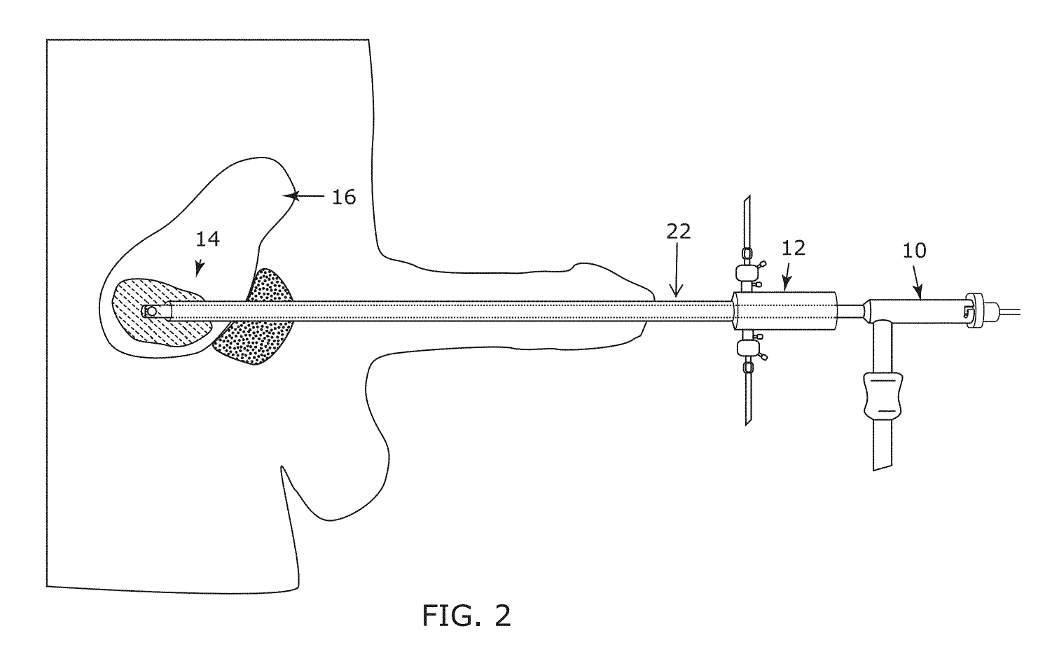

The cystothrombus evacuator 10 includes an aspiration tube 18 providing a maceration/homogenization tip for simultaneously aspirating (drawing by suction from the urinary bladder 16) blood clots 14 and morcellating said blood clots (dividing the blood clots into fragments or pieces for removal). The slender aspiration tube 18 may be nested within the cystoscope sheath 22; the slender aspiration tube 18 may be dimensioned accordingly, for example by having a diameter of approximately 5 millimeters in diameter. The aspiration tube 18 may be fluidly connected to an external suction source. Within a distal end of the aspiration tube 18, a motor driven element 24 may be provided with the operatively associated maceration/homogenization blade 82. While suction is applied, the motor driven element 24 rotates, mincing up the clot 14 and allowing it to be quickly removed, as illustrated in FIG. 2. The motor driven element 24 may be a rod, cable, or the like.

The distal end of the aspiration tube 18 may fluidly communicate to a suction intake 28 before terminating in an end cap 26. A proximal end of the aspiration tube 18 may be fluidly coupled to an aspirator chamber 30 and Touhy Borst valve 20 or equivalent. The motor driven element 24 may extend from the maceration/homogenization blade 82 near the distal end of the aspiration tube 18 to a drive mount assembly and operatively associated cable drive 34, as illustrated in FIG. 1. The cystothrombus evacuator 10 may also provide a handle 36 for manually operation.

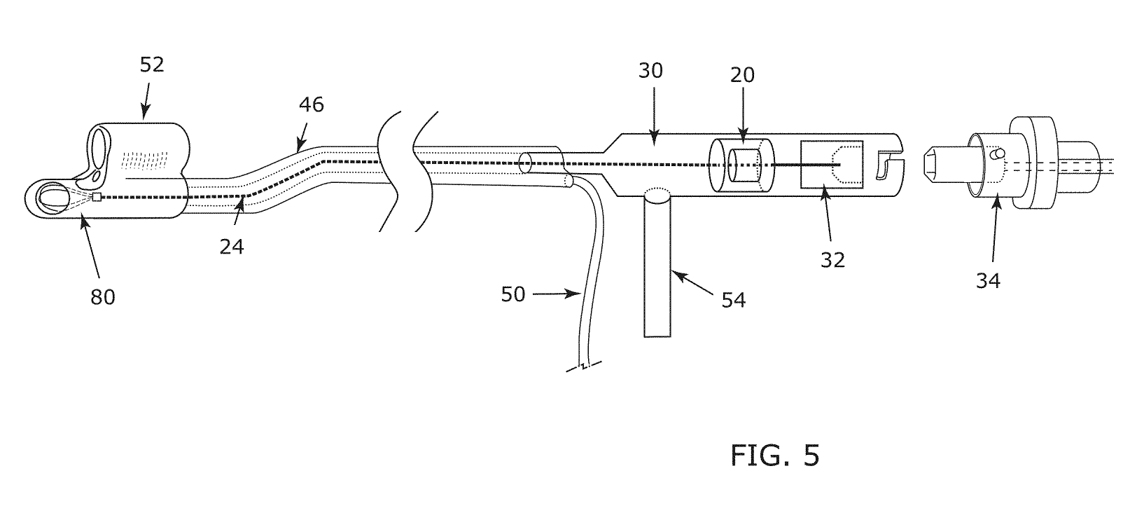

Referring to FIGS. 5 through 10, the gastroentero-thrombus evacuator 30 may be operatively couplable to an endoscope 42 (an illuminated optical, typically slender and tubular instrument introduced inside a patient's body to look therein). The operative coupling of the gastroentero-thrombus evacuator 30 and the endoscope device 42 enables the emergent removal of organized blood clots 14 from the human upper digestive system 44, while rapidly permitting endoscopic visualization and treatment of the bleeding site.

The gastroentero-thrombus evacuator 30 simultaneously aspirates and morcellates blood clots 14, through a flexible double lumen tube 46 attached alongside a standard flexible endoscope 42. The double lumen tube 46 may be attached to the endoscope 42 by way of a coupling device 52 as well as a removably connector 62, such as a hook and loop fastener. The coupling device may provide an endo opening 84 for operatively engaging a distal end of an endoscope 42. In certain embodiments, the end of the endoscope may be a video endoscope 40, as illustrated in FIG. 10. A first lumen 48 of the double lumen tube is fluidly connected to an external suction source via a suction port 54. Within this first lumen 48 may be a motor driven element 24 with an operatively associated maceration/homogenization tip 80 and blade 82. While external suction is applied, the motor driven element 24 rotates, mincing up the clot 14, permitting aspiration. The second lumen 50 of the double lumen tube 46 is fluidly connected—possibly via an inlet/outlet flushing port 56/58—to an external reservoir of sterile saline solution, for irrigation/flushing, to assist the aspiration of clot fragments.

Like the cystothrombus evacuator 10, the distal end of the double lumen tube 46 may fluidly communicate to a suction intake 28, though the suction intake 28 may be provided in a coupling device 52. The coupling device 52 may provide a coupling cavity 86 fluidly interconnecting the suction intake 28 and the first and second lumens 48 and 50. In certain embodiments, the maceration blade 82 may be disposed at least in part in the coupling cavity 86. A proximal end of the double lumen tube 46 may be fluidly coupled to an aspirator chamber 30 and Touhy Borst valve 20 or equivalent. The motor driven element 24 may extend from the maceration/homogenization blade 82 near the distal end of the second lumen 50 to a drive mount assembly 32 and operatively associated cable drive 34, as illustrated in FIG. 5. The gastroentero-thrombus evacuator 30 may also provide a video input device 60.

The gastroentero-thrombus evacuator 30 does not interfere with the internal working channels or lens of the endoscope 42, because it functions over the endoscope 42, not through it.

By increasing morcellation performance through combination optimized suction nozzles, sharpened maceration blades, and sturdier rotational elements, the gastroentero-thrombus evacuator could be used for other more vigorous applications such as removal of tumor, bezoars or feces.

It should be understood, of course, that the foregoing relates to exemplary embodiments of the invention and that modifications may be made without departing from the spirit and scope of the invention as set forth in the following claims.

What is claimed is:

1. A thrombus evacuator device, comprising:
an aspirator tube extending from a distal end to a proximal end;
a suction intake fluidly communicating with the distal end;
a maceration blade provided within the aspirator tube adjacent to the suction intake; and
rotational element operatively associated to the maceration blade for morcellating blood clots urged through the suction intake,
wherein the aspirator tube is dimensioned to slidably nest in a cystoscope sheath adapted to be inserted into a human urethra.

2. The thrombus evacuator device of claim 1, further including an end cap removably attached to the distal end, the end cap providing the suction intake.

3. The thrombus evacuator device of claim 2, further providing a drive assembly connected to the proximal end, the drive assembly operative associated to the rotational element.

4. The thrombus evacuator device of claim 3, the maceration blade comprising a whisk configuration.

5. The thrombus evacuator device of claim 1, further comprising a suction through the aspirator tube.

6. A thrombus evacuator device, comprising:
a double lumen tube extending from a distal end to a proximal end, the double lumen tube providing non-fluidly connected first and second lumens;
a coupling device removably attached to the distal end, the coupling device providing an endo opening for operatively engaging a distal end of an endoscope; the coupling device providing a suction intake fluidly communicating with the first lumen; the coupling device providing a flushing outlet fluidly communicating with the second lumen;
a maceration blade provided adjacent to the suction intake; and
rotational element operatively associated to the maceration blade for morcellating blood clots urged through the suction intake, the rotational element extending through the second lumen.

7. The thrombus evacuator device of claim 6, further providing a drive assembly connected to the proximal end, the drive assembly operative associated to the rotational element.

8. The thrombus evacuator device of claim 7, the maceration blade comprising a whisk configuration.

9. The thrombus evacuator device of claim 6, further comprising a suction through the first lumen.

10. The thrombus evacuator device of claim 6, the coupling device providing a coupling cavity fluidly interconnecting the suction intake and the first and second lumens.

11. The thrombus evacuator device of claim 10, the maceration blade disposed at least in part in the coupling cavity.

12. A thrombus evacuator device, comprising:
a double lumen tube extending from a distal end to a proximal end, the double lumen tube providing non-fluidly connected first and second lumens;
a coupling device removably attached to the distal end, the coupling device providing an endo opening for operatively engaging a distal end of an endoscope; the coupling device providing a coupling cavity fluidly interconnecting the first and second lumens; the coupling device providing a suction intake fluidly communicating with the coupling cavity; the coupling device providing a flushing outlet fluidly communicating with the coupling cavity;
a maceration blade provided adjacent to the suction intake; and
rotational element operatively associated to the maceration blade for morcellating blood clots urged through the suction intake, the rotational element extending through the second lumen.

13. The thrombus evacuator device of claim 12, further comprising a suction through the first lumen.
